(19) United States
(12) Patent Application Publication
(10) Pub. No.: US 2025/0261981 A1
(43) Pub. Date: Aug. 21, 2025

(54) BONE FIXATION SYSTEM AND METHODS OF USE

(71) Applicant: DUET SPINE HOLDINGS, LLC, Winona Lake, IN (US)

(72) Inventors: David WALSH, Reading, MA (US); Richard G. FESSLER, Lake Forest, IL (US)

(73) Assignee: DUET SPINE HOLDINGS, LLC, Winona Lake, IN (US)

(21) Appl. No.: 19/204,084

(22) Filed: May 9, 2025

Related U.S. Application Data
(63) Continuation of application No. PCT/US2023/079466, filed on Nov. 13, 2023.
(60) Provisional application No. 63/424,367, filed on Nov. 10, 2022.

(51) Int. Cl. A61B 17/86; A61B 17/00; A61B 17/70

(52) U.S. Cl. CPC A61B 17/8685; A61B 17/70; A61B 17/8605; A61B 17/8625; A61B 17/864; A61B 2017/00477

(57) ABSTRACT

A bone fixation system including a first fastener, a second fastener, and a set screw. The first fastener may include a first shaft and a first head. The first head may define a passageway and may have a passageway axis. The first head may include an interior surface defining a curved seat, a front surface, and a U-shaped surface connecting each side of the front surface. The second fastener of the bone fixation system may include a second shaft and a second head shaped for disposal within the passageway such that the curved surface of the second head may contact the seat of the first head and may be pivotable about the curved surface. The set screw of the bone fixation system may secure the second head to lock the second fastener in a fixed, non-pivotable position.

### CROSS REFERENCE TO RELATED APPLICATION

This application is a continuation of International Application No. PCT/US2023/079466 filed Nov. 13, 2023, which claims benefit of priority of U.S. Provisional Patent Application No. 63/424,367, filed Nov. 10, 2022, and entitled “Bone Fixation System and Methods of Use,” the disclosure of which is hereby incorporated herein by reference in its entirety.

### FIELD OF INVENTION

The present disclosure relates generally to general surgery, orthopedic and neurosurgical implants used for insertion within a patient's vertebrae. More specifically, but not exclusively, the present disclosure concerns bone fixation systems for implantation into a patent's spine to maintain or re-establish proper spacing and alignment of the spine.

### BACKGROUND

Spinal deformities may result from disease, age or trauma causing destabilization of the spine. To correct destabilization of a patent's spine, posterior fusion device systems may be used. The posterior fusion device systems that are currently available are designed to be applicable to single and multiple level stabilizations using a rod extending between adjacent bone screws for strength. These posterior fusion device systems and the instrumentation used for insertion into a patient's spine are extensive, complicated and expensive and susceptible to loosening of the rods and screws.

### SUMMARY

Aspects of the present disclosure provide bone fixation systems, including at a single level or multiple levels, and methods that can maintain or re-establish anatomic spacing within a patent's spine.

Some embodiments of the present disclosure may include a bone fixation system including a first fastener, a second fastener, and a set screw. The first fastener may include a first shaft and a first head. The first shaft may include a distal end, a proximal end, a first longitudinal axis, and a first neck disposed at the proximal end. The first neck may be unthreaded. The first head may be disposed at the proximal end of the first shaft and may define a passageway extending from a first end to a second end and having a passageway axis. The first head may also include an interior surface along the passageway, wherein the interior surface defines a curved seat, a front surface disposed between the first neck of the first shaft and the first end of the first head, and a U-shaped surface connecting each side of the front surface. The U-shaped surface may comprise a first side surface, a second side surface, and a curved surface connecting the first and second side surfaces. The first and second side surfaces may be generally planar and are generally aligned with the passageway axis. The front surface may be generally aligned with the first longitudinal axis. The first longitudinal axis and the passageway axis may be obliquely angled. The second fastener of the bone fixation system may include a second shaft and a second head. The second shaft may include a distal end, a proximal end, a second longitudinal axis, and a second neck disposed at the proximal end. The second neck may be unthreaded. The second head may be disposed at the proximal end of the second shaft and may be bulbous and have a curved surface. The second head may be shaped for disposal within the passageway such that the curved surface of the second head may contact the seat of the first head and may be pivotable about the curved surface. The set screw of the bone fixation system may be introducible into the passageway along the passageway axis to secure the second head of the second fastener in the passageway between the seat and the set screw and to lock the second fastener in a fixed, non-pivotable position.

In some embodiments, a first edge between the first side surface and the front surface may be rounded and a second edge between the second side surface and the front surface may be rounded. In some embodiments, the first fastener may be threaded between the unthreaded first neck and the distal end. In some embodiments, the threading of the first fastener may comprise a first portion and a second portion, wherein the first portion has a higher thread pitch than the second portion. In some embodiments, an intersection between the first neck and the threading on the first shaft may be at least one of rounded or tapered. In some embodiments, the second fastener may be threaded between the unthreaded second neck and the distal end. In some embodiments, the second longitudinal axis may be parallel to the passageway axis and, in other embodiments, the second longitudinal axis may be obliquely angled relative to the passageway axis. In some embodiments, the second fastener may be pivotable such that the second longitudinal axis can be angled in a range of 0 degrees to 15 degrees relative to the passageway axis.

Some embodiments of the present disclosure may include a bone fixation system including a first fastener, a second fastener, and a set screw. In some embodiments, the first fastener may include a first shaft and a first head. The first shaft may include a threaded distal end, a proximal end, a first longitudinal axis, and a first neck disposed at the proximal end. The first neck maybe e unthreaded. The first head may be disposed proximal to the neck, may define a passageway extending between a first passage end and a second passage end, and may have a passageway axis obliquely angled relative to the first longitudinal axis. The first head may comprise a seat disposed along the passageway. An outer profile of the first head may not match an inner profile of the first head. The inner profile may be cylindrical and the outer profile may comprise at least one flat surface. The second fastener of the bone fixation system may include a second shaft and a second head. The second shaft may include a distal end, a proximal end, a second longitudinal axis, and a second neck disposed at the proximal end. The second neck may be unthreaded. The second head may be disposed proximal to the second neck, may be bulbous, and may have a curved surface. The second head may be shaped for disposal within the passageway such that the curved surface of the second head contacts the seat of the first head and is pivotable about the curved surface. The set screw of the bone fixation system may be introducible into the passageway along the passageway axis to secure the second head of the second fastener in the passageway between the seat and the set screw and to lock the second fastener in a fixed, non-pivotable position.

In some embodiments, the outer profile may comprise a front surface disposed between the first neck of the first shaft and a top surface of the first head and a U-shaped surface connecting each side of the front surface. The U-shaped surface may comprise a first side surface, a second side surface, and a curved surface connecting the first and second side surfaces, and at least one of the front surface, the first side surface, and the second side surface may be the at least one flat surface. In some embodiments, a first edge between the first side surface and the front surface may be rounded and a second edge between the second side surface and the front surface may be rounded. In some embodiments, the first fastener may be threaded between the unthreaded first neck and the distal end. In some embodiments, an intersection between the first neck and the threading on the first shaft may be at least one of rounded or tapered. In some embodiments, the second longitudinal axis may be parallel to the passageway axis and, in other embodiments, the second longitudinal axis may be obliquely angled relative to the passageway axis. In some embodiments, the second fastener may be pivotable such that the second longitudinal axis is angled in a range of 0 degrees to 15 degrees relative to the passageway axis.

Some embodiments of the present disclosure may include a method of implanting a bone fixation system. The method may include the steps of: introducing a first fastener into a first bone; introducing a second fastener into a second bone; implanting the first fastener; implanting the second fastener through the passageway at a first angle, such that the first angle is one of parallel to the passageway axis or obliquely angled relative to the passageway axis; and, securing the second head of the second fastener in the passageway using a set screw to lock the second fastener in a fixed, non-pivotable position. In some embodiments, the first fastener may include a first shaft and a first head. The first shaft may include a distal end, a proximal end, a first longitudinal axis, and a first neck disposed at the proximal end. The first neck may be unthreaded. The first head may be disposed at the proximal end of the first shaft, may define a passageway extending from a first end to a second end, and may have a passageway axis. The first head may include an interior surface along the passageway, such that the interior surface defines a curved seat, a front surface disposed between the first neck of the first shaft and the first end of the first head, and a U-shaped surface connecting each side of the front surface. The U-shaped surface may include a first side surface, a second side surface, and a curved surface connecting the first and second side surfaces. The first and second side surfaces may be generally planar and may be generally aligned with the passageway axis. The front surface may be generally aligned with the first longitudinal axis. The first longitudinal axis and the passageway axis may be obliquely angled. In some embodiments, the second fastener may include a second shaft and a second head. The second shaft may include a distal end, a proximal end, a second longitudinal axis, and a second neck disposed at the proximal end. The second neck may be unthreaded. The second head may be disposed at the proximal end of the second shaft, and may be a bulbous head having a curved surface. The second head may be shaped for disposal within the passageway such that the curved surface of the second head contacts the seat of the first head and is pivotable about the curved surface.

In some embodiments, a first edge between the first side surface and the front surface may be rounded and a second edge between the second side surface and the front surface may rounded. In some embodiments, the first fastener may also include a threaded portion between the unthreaded first neck and the distal end and an intersection between the first neck and the threaded portion. The intersection may be at least one of rounded or tapered.

These and other objects, features and advantages of this disclosure will become apparent from the following detailed description of the various aspects and principles of the disclosure taken in conjunction with the accompanying drawings.

### BRIEF DESCRIPTION OF THE DRAWINGS

The accompanying drawings, which are incorporated in and constitute a part of the specification, illustrate embodiments of the disclosure and, together with the detailed description herein, serve to explain the principles of the disclosure. The drawings are only for purposes of illustrating examples and are not to be construed as limiting the disclosure. It is emphasized that, in accordance with the standard practice in the industry, various features are not drawn to scale. In fact, the dimensions of the various features may be arbitrarily increased or reduced for clarity of discussion.

FIG. 1 illustrates a side view of one embodiment of a bone fixation system implanted in a spine, in accordance with one or more aspects of the present disclosure;

FIG. 2 illustrates a perspective view of one embodiment of a bone fixation system, in accordance with one or more aspects of the present disclosure;

FIG. 3A illustrates a cross-sectional view of the assembled bone fixation system shown in FIG. 2, in accordance with one or more aspects of the present disclosure;

FIG. 3B illustrates an exploded, cross-sectional view of the bone fixation system shown in FIG. 2;

FIG. 4A illustrates a perspective view of one embodiment of a first fastener of the bone fixation system of FIG. 2, in accordance with one or more aspects of the present disclosure;

FIG. 4B illustrates a side view of the first fastener of FIG. 4A, in accordance with one or more aspects of the present disclosure;

FIG. 4C illustrates a partial cross-sectional view of the first fastener of FIG. 4A, in accordance with one or more aspects of the present disclosure;

FIG. 4D illustrates a top view of the first fastener of FIG. 4A, in accordance with one or more aspects of the present disclosure;

FIG. 5 illustrates a side view of one embodiment of a second fastener of the bone fixation system of FIG. 2, in accordance with one or more aspects of the present disclosure;

FIG. 6A illustrates a perspective view of one embodiment of a set screw of the bone fixation system of FIG. 2, in accordance with one or more aspects of the present disclosure;

FIG. 6B illustrates a cross-sectional view of the set screw of FIG. 6A, in accordance with one or more aspects of the present disclosure;

FIG. 7A illustrates a perspective view of one embodiment of an inserter instrument coupled to the bone fixation system of FIG. 2, in accordance with one or more aspects of the present disclosure;

FIG. 7B illustrates a perspective view of the inserter instrument of FIG. 7A, in accordance with one or more aspects of the present disclosure;

FIG. 8 is a flow chart that illustrates a surgical method for implanting the bone fixation system of FIG. 2, in accordance with one or more aspects of the present disclosure; and

FIG. 9 illustrates an enlarged cross-sectional view of the head of the primary fastener of the bone fixation system of FIG. 2, in accordance with one or more aspects of the present disclosure.

### DETAILED DESCRIPTION OF THE INVENTION

Generally stated, disclosed herein are different embodiments and examples of a bone fixation system. The embodiments and examples described herein of a bone fixation system constructed in accordance with one or more aspects of the present disclosure avoid the need for using a rod to connect adjacent pedicle screws typically used in back surgery. Without use of a connecting rod, there are less parts involved and less movable parts and less chance of movement of the fusion system or loosening of its components after surgery with a bone fixation system constructed in accordance with one or more principles or aspects of the present disclosure. A bone fixation system constructed in accordance with one or more principles or aspects of the present disclosure also does not rely on a connecting rod for strength. Further, surgical methods for inserting the bone fixation systems are discussed.

In this detailed description and the following claims, the words proximal, distal, anterior, posterior, medial, lateral, superior, inferior, cephalad and caudally are defined by their standard usage for indicating a particular part of a bone or implant according to the relative disposition of the natural bone or directional terms of reference. For example, “proximal” means the portion of an implant nearest the insertion instrument, while “distal” indicates the portion of the implant farthest from the insertion instrument. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure, “cephalad” means a direction toward the head and “caudally” means a direction toward the inferior part of the body.

In general, the embodiments and examples described herein of a bone fixation system constructed in accordance with one or more principles or aspects of the present disclosure include a primary screw or fastener and a secondary screw or fastener. The primary fastener may include a shaft and a head angled relative to the shaft. In some embodiments, a set screw is used to retain the second fastener within the head of the primary fastener. The head of the primary fastener provides a seat for the second fastener to enable variable angulations of the secondary fastener relative to the primary fastener.

In general, the head of the primary fastener is shaped and angled relative to the shaft to enable insertion of the secondary fastener through it and into an adjacent pedicle, thereby eliminating the need for a rod or plate. In some embodiments, the head of the primary fastener is shaped to accommodate the physiology of the spine. In some embodiments, the head of the primary fastener may be shaped to minimize contact or interference with bone including, for example, the spinous process or transverse process. In some embodiments, the shape of the head of the primary fastener provides a low profile and less trauma to soft tissues when inserted, for example, via minimally invasive techniques.

In some embodiments, the head of the primary fastener is enlarged with respect to the shaft of the primary fastener. For example, the cross-sectional width of the enlarged head, taken normal to the longitudinal axis of the head, may be greater than the cross-sectional width of the shaft, taken normal to the longitudinal axis of the shaft. In some embodiments, the head may be shaped and manufactured to provide support for the bone fixation system. For example, the head may be shaped to provide additional strength and stress resistance to the bone fixation system. The head may also be shaped to withstand the cyclical loading that a patient's spine generally undergoes in daily life.

Moreover, the head may be angled relative to the shaft of the primary fastener. The position of the head of the primary fastener ensures closest proximity of the head to bone, thereby maximizing strength of the system. The angulation of the head relative to the shaft of the primary fastener enables appropriate direction of the secondary fastener to engage a second vertebra or pedicle, thus eliminating the need for a rod or plate. A set screw may be inserted into the head of the primary fastener to secure the secondary fastener to the primary fastener in a particular angular relationship. By minimizing the number of parts, the risk of failure through, for example, loosening of set screws to tulip heads in conventional bone fixation systems is reduced.

In general, the head of the primary fastener and a portion of the second fastener are configured to reside outside bone to allow for trajectory of the secondary fastener to engage an adjacent pedicle or span multiple adjacent vertebrae levels. The head of the primary fastener is incorporated partially into the longitudinal axis of the shaft of the primary fastener to minimize the overall size of the head, thereby decreasing soft tissue trauma on insertion and long-term irritation of adjacent muscle. The angulated head of the primary fastener enables, for example, in line insertion of a primary fastener screwdriver, and insertion of a secondary fastener and screwdriver through the same external entrance of the primary fastener during insertion. Moreover, part or all of the exposed shaft of the primary and/or secondary fastener may be unthreaded to minimize soft tissue trauma and long-term irritation of adjacent muscle.

The bone fixation system described herein may also include a tapering or a gradually increasing thickness of the shaft of the primary fastener as it approaches the head. This tapering or increased thickness at a region of greatest stress on the primary fastener reduces failure through fracture of the primary fastener and enhances the fastener to bone interface thereby providing enhanced stability by increasing compressive load.

The combination of the primary fastener and secondary fastener described by the examples herein enables insertion of the bone fixation system via either an MIS or OPEN technique without the need for additional instrumentation. The system design relies on angulation of two fasteners relative to each other for stability and minimizes the need for additional instrumentation or components, such as, for example, rods and multiple screw lengths and thicknesses. The design of the bone fixation system described herein combines the resistance to “pull out” imparted by the threads of the fasteners with that created by the angulation of the fasteners relative to each other, thereby making pull out virtually impossible.

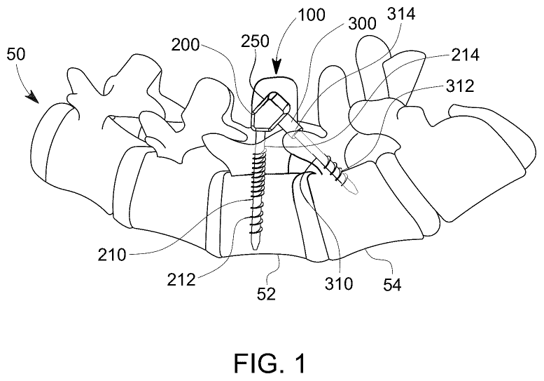

Referring to the drawings, FIG. 1 illustrates an exemplary embodiment of a bone fixation system 100 implanted into the spine 50. In the illustrated example, the bone fixation system 100 is implanted into one level of the spine 50. The bone fixation system 100 may include a primary fastener 200 and a secondary fastener 300. The primary fastener 200 may be implanted into a first vertebra 52 and the secondary fastener 300 may be implanted into a second vertebra 54 (where the first vertebra 52 and the second vertebra 54 are shown as partially transparent in FIG. 1). The first vertebra 52 and second vertebra 54 may be neighboring vertebra.

The primary fastener 200 may comprise a shaft 210 and a head 250. The shaft 210 may have a threaded portion 212 and an unthreaded portion or neck 214. The threaded portion 212 may be implanted into the first vertebra 52 such that part or all of the threaded portion 212 is implanted in the bone. The unthreaded neck 214 may not be implanted in the first vertebra 52 and may be disposed above the first vertebra 52. In some embodiments, part or all of the unthreaded neck 214 may be implanted into bone.

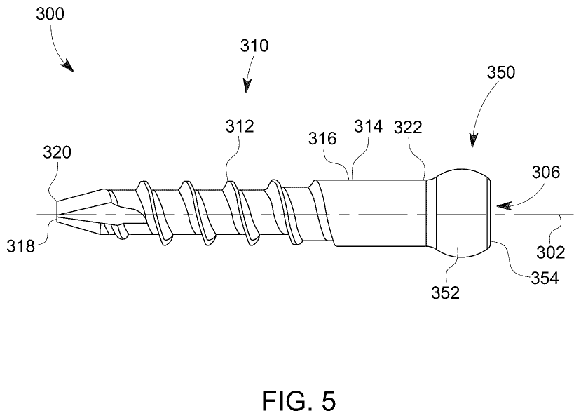

The secondary fastener 300 may comprise a shaft 310 and a head 350 (not shown in FIG. 1; shown in FIG. 5). The shaft 210 may have a threaded portion 312 and an unthreaded portion or neck 314. The secondary fastener 300 may be inserted through the head 250 of the primary fastener 200 such that the head 350 of the secondary fastener 300 is seated in the head 250 of the primary fastener 200, as described in more detail below. The threaded portion 312 of the secondary fastener 300 may be implanted in the second vertebra 54 such that part or all of the threaded portion 312 is implanted in the bone. The unthreaded neck 314 may not be implanted in the second vertebra 54 and may be disposed above the second vertebra 54. In some embodiments, part or all of the unthreaded neck 314 may be implanted into bone. In some embodiments, the threading 212, 312 and the angling of the fasteners 200, 300 relative to each other may prevent the bone fixation system 100 from backing out of the bone.

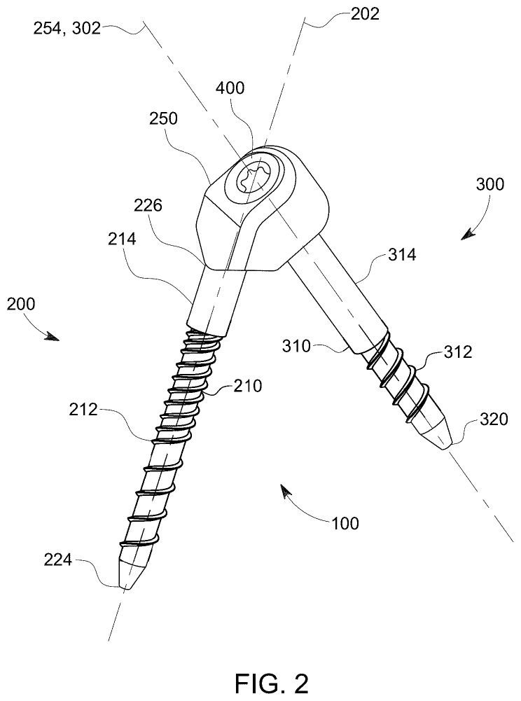

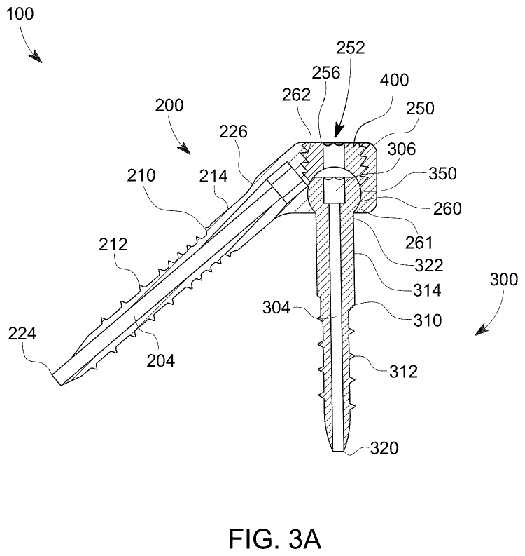

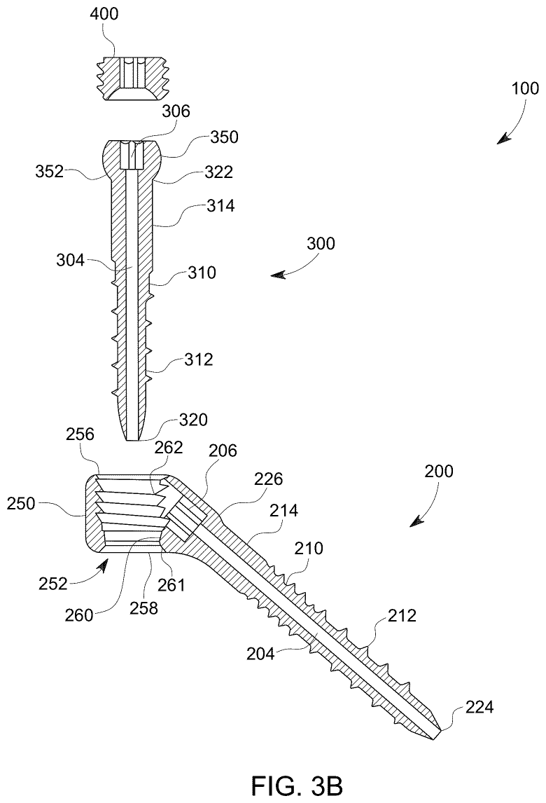

FIGS. 2, 3A, and 3B illustrate the bone fixation member 100 according to some embodiments of the present disclosure. FIG. 2 illustrates a perspective view of the bone fixation member 100 according to some embodiments. FIGS. 3A and 3B illustrate a cross-sectional view of the bone fixation member shown in FIG. 2 in an assembled and exploded configuration, respectively. The primary fastener 200, which may also be referred to as a first fastener, a primary bone anchor, or a first bone anchor, may include a shaft 210, a head 250, and a longitudinal axis 202. The shaft 210 may have a distal end 224 and a proximal end 226. The shaft 210 may be aligned with the longitudinal axis 202 and may have a threaded portion 212 and an unthreaded portion or neck 214. The head 250 may be enlarged relative to the shaft 210 and may have a passageway 252 or opening that passes through it. The head 250 may include at least two planar outer side surfaces 278, 280 to facilitate coupling with an insertion instrument. The at least two planar side surfaces 278, 280 may be positioned on opposite sides of the head 250. An arcuate outer surface 282 may be present to connect the two planar side surfaces 278, 280 on the periphery of the head 250. The arcuate surface 282 may be positioned opposite from the neck 214 of the head 250. The passageway 252 may have a passageway axis 254. In some embodiments, the passageway axis 254 may be obliquely angled with respect to the longitudinal axis 202 of the primary fastener 200. For example, in some embodiments, the angle between the longitudinal axis 202 and the passageway axis 254 may be 5°, 10°, 15°, 20°, 25°, 30°, 35°, 40°, 45°, 50°, 55°, 60°, 65°, 70°, 75°, 80°, 85°, or 90°. In some embodiments, the angle may preferably be in a range of about 40° to 60°, and in some implementations, at approximately 50°. The passageway 252 defines an interior cavity or space having an inner surface 262 extending from a first end 256 to a second end 258. The passageway 252 may be sized and configured to receive and allow pass through of at least a portion of second fastener 300 or other spinal connection element to anchor the system 100 to, for example, a second bone or vertebrae 54. The passageway 252 may also be sized and configured to retain or secure at least a portion of head 350 of second fastener 300.

The second fastener 300, which also may be referred to as a second fastener, a secondary bone anchor, or a second bone anchor, may include a shaft 310, a head 350 (not shown in FIG. 2), and a longitudinal axis 302. The shaft 310 of the secondary fastener 300 may have a distal end 320 and a proximal end 322. The shaft 310 of the secondary fastener 300 may include a threaded portion 312 and an unthreaded portion or neck 314. The secondary fastener 300 may be inserted through the passageway 252 of the head 250 of the primary fastener 200 such that the head 350 may be seated in the head 250 of the primary fastener 200. A set screw 400 may be introduced into the passageway 252 of head 250 of the primary fastener 200 to secure the head 350 of the secondary fastener 300 into the head 252 of the primary fastener 200. In some embodiments, as shown in the illustrated embodiment, the longitudinal axis 302 of the secondary fastener 300 may be coaxial with the passageway axis 254. However, in other embodiments, the longitudinal axis 302 may be obliquely angled with respect to the passageway axis 254, as described in more detail below.

In some embodiments, as shown the illustrated embodiment, the primary fastener 200 may include a cannula 204 through the shaft 210. The cannula 204 may intersect with the passageway 252 of the head 250. There may be a tool engagement feature 206 at the top of the cannula 204. Additionally, in some embodiments, as shown in the illustrated embodiment, the secondary fastener 300 may have a cannula 304 that passes from the top of the head 350 through the distal end 320 of the shaft 310. There may be a tool engagement feature 306 at the top of the cannula 304.

The passageway 252 has a first end 256 and a second end 258 opposite the first end 256. There may be a seat 260 formed in the interior surface 262 of the passageway 252 proximate the second end 258 of the passageway 252. The seat 260 may be shaped to contact the bottom of the head 350 of the secondary fastener 300. The seat 260 may extend along at least another portion of interior surface 262 towards second end 258. In one example, seat 260 may be formed by interior surface 262 tapering inward towards longitudinal axis 302 as it approaches second end 258.

The head 350 of the secondary fastener 300 may be enlarged with respect to the shaft 310. The head 350 may be bulbous, spherical, semi-spherical or otherwise rounded. The head 350 may have a curved bottom 352 that curves to a flat top surface 354. In some embodiments, the seat 260 of the passageway 252 is shaped to match the curved bottom surface 352 of the head 350 of the secondary fastener 300. When the curved bottom surface 352 rests on the seat 260, the secondary fastener 300 may pivot about the curved bottom surface 352 on the seat 260. Thus, the secondary fastener 300 may pivot, spin, slide, rotate, or otherwise move through a variety of positions against the seat 260. In some embodiments, the longitudinal axis 302 of the secondary fastener 300 may be obliquely angled with respect to the passageway axis 254. This configuration allows a range of motion along several different axes, e.g. multi-directional movement or rotation or angulation, of secondary fastener 300 relative to the passageway axis 254. For example, a curved bottom surface 352 of secondary fastener 300 mating with a mating concave-shaped seat 260 enables a variation of angulation to accommodate pedicle screw insertion in variable anatomy. For example, the secondary fastener 300 may pivot through a variety of angles in a range of 0° to 5°, 10°, 15°, 20°, 25°, 30°, 35°, 40°, 45°, 50°, 55°, 60° relative to the passageway axis 254. In some embodiments, the secondary fastener 300 may pivot through a variety of angles in a range of 0° to 15° relative to the passageway axis 254.

In some embodiments, the secondary fastener 300 may be coaxial with the passageway axis 254. In other embodiments, the secondary fastener 300 may be obliquely angled with respect to the passageway axis 254. Moreover, in some embodiments, the secondary fastener 300 may be obliquely angled relative to the longitudinal axis 202 of the primary fastener 200. In other embodiments, the secondary fastener 300 may be parallel to the longitudinal axis 202 of the primary fastener 200.

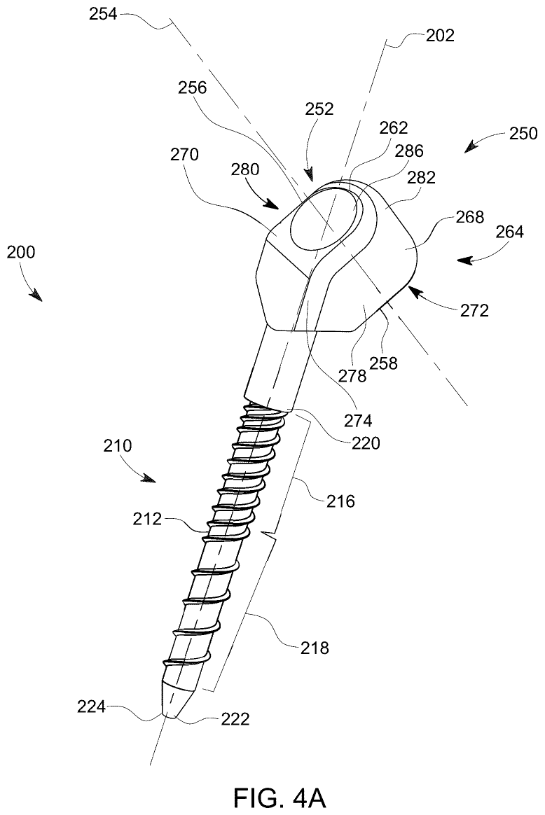

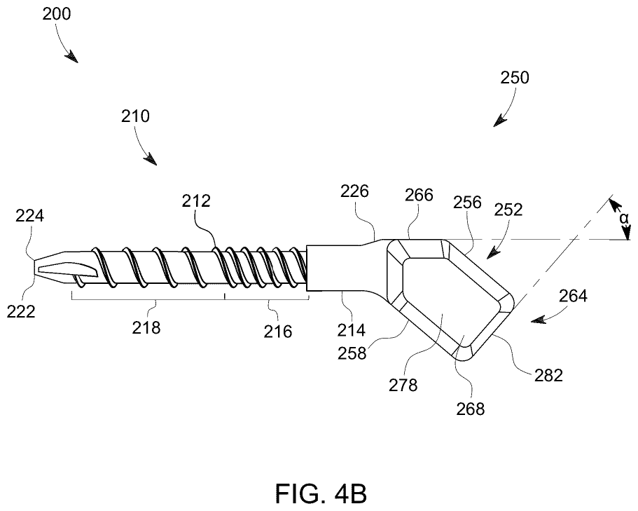

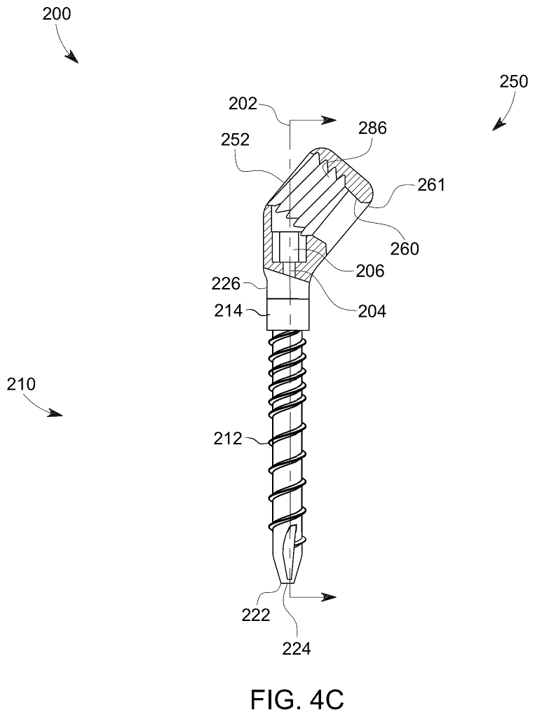

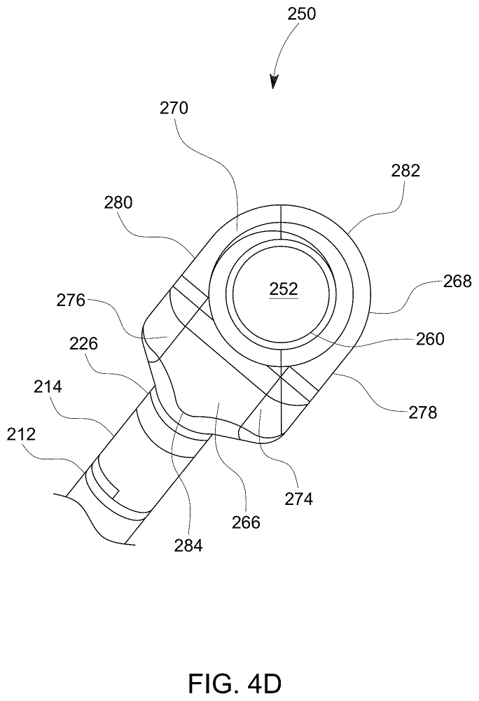

FIGS. 4A-4D illustrate various views of the primary fastener 200 in FIG. 2. FIG. 4A is a perspective view, FIG. 4B is a side view, FIG. 4C is a partial cross-sectional side view, and FIG. 4D is a top view of the primary fastener 200. In the illustrated embodiment, the shaft 210 of the primary fastener 200 includes a threaded portion 212 and an unthreaded portion or neck 214.

The threaded portion 212 may have any appropriate profile. In the illustrated embodiment, the threaded portion 212 has a first portion 216 and a second portion 218. The first portion 216 may have a lower thread pitch than the second portion 218. In some aspects, the lower thread pitch may make the first portion 216 better suited for cortical bone and the higher thread pitch may make the second portion 218 better suited for cancellous bone. In other embodiments, the threaded portion 212 may have more than two portions, which may each have different thread pitches or one or more portions may have the same thread pitch. In other embodiments, the threaded portion 212 may not have more than one portion and may instead have one portion of the same thread pitch. In some embodiments, the threaded portion 212 may be single lead or dual lead. In other embodiments, the shaft 210 may be threaded along its entire length and may have no unthreaded portion.

The shaft 210 may also have an intersection 220 between the threaded portion 212 and the unthreaded neck 214. The intersection 220 may be the thread runout of the threaded portion 212. In some embodiments, the thread runout 220 may be gradual. The thread runout 220 may be gradual over a range of about 0.5 to 6 full rotations. In some implementations, the thread runout is in a range of about 0.5 to 2 full rotations. In some implementations, the thread runout includes a taper angle in a range of about 3 to 30 degrees. In some implementations, the thread runout includes a taper angle in a range of about 5-10 degrees.

The shaft 210 may be tapered from the proximal end 226 to the distal end 224 and may come to a point 222 at the distal end 224. In some embodiments, only the threaded portion 212 or unthreaded neck 214 may be tapered. In other embodiments, the shaft 210 may not be tapered, but may have discrete sections or steps where the width narrows.

The head 250 of the primary fastener 200 may be disposed at the proximal end 226 of the unthreaded neck 214. The passageway 252 or interior surface 262 of the head 250 may be generally cylindrically shaped as in the illustrated embodiment. There may be a seat 260 formed in the interior surface 262. The seat 260 may be curved to match the shape of the head 350 of the secondary fastener 300. There may also be a skirt 261 extending outward from the seat 260 to the second end 258 of the passageway 252. In some embodiments, the skirt 261 may be curved or flat. The skirt 261 may provide additional space for the unthreaded neck 314 of the secondary fastener 300 to move or rotate as the secondary fastener 300 pivots about the seat 260. Thus, the skirt 261 may provide an added pivoting range of motion.

As shown in FIG. 9, the pivoting range of motion (See A in FIG. 9) relative to the passageway axis 254 in one direction may be 10° to 15° or preferably 13° with a full range of motion from 20° to 30° with a preferable total range of motion of 26° relative to the passageway axis 254. In other embodiments, the skirt 261 may not extend outward, but may extend further inward or may extend straight down from the bottom of the seat 260 to the second end 258 of the passageway 250. In some embodiments, there is no skirt 261 and the seat 260 is disposed along the second end 258 of the passageway 252.

The head 250 may have an exterior or outer surface 264. In some embodiments, the exterior surface 264 of the head 250 does not match the interior surface 262. In the illustrated embodiments, the exterior surface 264 is comprised of a front surface 266 and a back region 268. The front surface 266 extends from the proximal end 226 of the unthreaded neck 214 to the top surface 270 of the head 250. The back region 268 may be generally U-shaped and may connect a first side edge 274 of the front surface 266 with a second side edge 276 of the front surface 266. The back region 268 may have a first side surface 278 and a second side surface 280 connected by a curved surface 282. In some embodiments, the first side surface 278 and second side surface 280 may be connected by a flat surface.

In some embodiments, the first side surface 278 and the second side surface 280 are generally planar and parallel to each other. In some embodiments, the edges between the front surface 266 and the back region 268 are rounded or curved. In other embodiments, the edges come to a pointed corner or are flat and angled at a chamfer. In some embodiments, the front surface 266 may extend from the side edges 274, 276 and come to a point 284 at the bottom along the proximal end 226 of the unthreaded neck 214.

In some embodiments, the front surface 266 is arranged so that it is generally aligned with the longitudinal axis 202 of the primary fastener 200 such that the longitudinal axis 202 and the plane of the front surface 266 are parallel to each other. In some embodiments, the back region 268 is generally aligned with the passageway axis 254 such that the surfaces 278, 280, 282 are generally parallel to the passageway axis 254. Thus, a line or boundary along the front surface 266 and a line or boundary along the back region 268 may be angled relative to each other, as shown in FIG. 4B at angle a. More specifically, FIG. 4B shows a boundary line along the curved surface 282 that intersects a boundary line along the front surface 266 and forms the angle α. In some embodiments, the angle a may be about 5°, 10°, 15°, 20°, 25°, 30°, 35°, 40°, 45°, 50°, 55°, 60° 65°, 70°, 75°, 80°, 85°, or 90°. In some embodiments, the angle may preferably be in a range of about 45° to 55°, and in some implementations, at approximately 50°. In other embodiments, the front surface 266 may be angled with respect to the longitudinal axis 202. In some embodiments, the back region 268 may be angled away from the passageway axis 254 from the top surface 270 to the bottom surface 272 of the head 250. In other embodiments, the back region 268 may be angled towards the passageway axis 254 from the top surface 270 to the bottom surface 272 of the head 250.

The head 250 may be shaped to improve the strength of the primary fastener 200. In some embodiments, the front 266 and back 268 surfaces are shaped to add support for the head 250 and unthreaded neck 214. The head 250 may also be shaped to accommodate the physiology of the spine including, for example, to minimize contact or interference with bone such as, for example, the spinous process or transverse process. The head 250 may also provide a low profile and less trauma to soft tissues when inserted. In some embodiments, the head 250 may be shaped to provide additional strength and stress resistance. For example, the head 250 may be shaped to withstand the cyclical loading that a patient's spine generally undergoes in daily life.

The interior surface 262 along the passageway 252 may have a threaded portion 286. The threaded portion 286 may extend partially or completely down the passageway 252. In some embodiments, the threaded portion 286 may be proximate the first end 256 of the passageway 252. The threaded portion 286 may engage threading on the set screw 400, as described in more detail below.

There may be a cannula 204 that passes through the shaft 210 of the primary fastener 200. The cannula 204 may open into and intersect the passageway 252 along the interior surface 262. The top of the cannula 204 may include a tool engagement feature 218. The cannula 204 and tool engagement feature 206 may be accessible through the first end 256 of the passageway 252. Thus, a guidewire may be inserted from the first end 256 of the passageway 256 through the cannula 204. Moreover, a tool, like for example a screwdriver, may be able to engage the tool engagement feature 206 through the first end 256 of the passageway 252 to implant, or screw, the primary fastener 200 into the bone. In some embodiments, the guidewire may be inserted through the cannula 204 of the primary fastener 200 and inserted into the desired location in the vertebra. Then, the shaft 210 of the primary fastener 200 may be implanted around the guidewire using a tool that engages the tool engagement feature 206. In some embodiments, the tool engagement feature 206 may be hexagonally or hexalobe shaped. However, in other embodiments, the tool engagement feature 206 may be any other appropriate shape including, triangular, square, rectangular, ovular, or pentagonal.

FIG. 5 illustrates a side view of the secondary fastener 300 of the bone fixation system 100 shown in FIG. 2. In the illustrated embodiment, the shaft 310 of the secondary fastener 300 includes a threaded portion 312 and an unthreaded portion or neck 314.

The threaded portion 312 may have any appropriate profile. In the illustrated embodiment, the threaded portion 312 has a single thread pitch. In some embodiments, the thread pitch of the threaded portion 312 of the secondary fastener 300 may be higher than the thread pitch of both threaded portions 216, 218 of the primary fastener 200. In other embodiments, the thread pitch of the threaded portion 312 of the secondary fastener 300 may be lower than the thread pitch of both threaded portions 216, 218 of the primary fastener 200 or may the same as the thread pitch of one of the threaded portions 216, 218. In some embodiments, the threaded portion 312 of the secondary fastener 300 may have more than one portion that may have the same or different thread pitches. In some embodiments, the threaded portion 312 of the secondary fastener 300 may be single lead or dual lead. In other embodiments, the shaft 310 may be threaded along its entire length and may have no unthreaded portion.

The shaft 310 may also have an intersection 316 between the threaded portion 312 and the unthreaded neck 314. The intersection 316 may be the thread runout of the threaded portion 312. In some embodiments, the thread runout 316 may be gradual. The thread runout 316 may be gradual over a range of about 0.5 to 6 full rotations. In some implementations, the thread runout is in a range of about 0.5 to 2 full rotations. In some implementations, the thread runout includes a taper angle in a range of about 3 to 30 degrees. In some implementations, the thread runout includes a taper angle in a range of about 5-10 degrees.

The shaft 310 may be tapered from the proximal end 322 to the distal end 320 and may come to a point 318 at the distal end 320. In some embodiments, only the threaded portion 312 or unthreaded neck 314 may be tapered. In other embodiments, the shaft 310 may not be tapered, but may have discrete sections where the width narrows.

The secondary fastener 300 may have a bulbous, spherical, or semispherical head 350. The head 350 may have a curved bottom surface 352 and a flat top surface 354.

The secondary fastener 300 may have a cannula 304 that passes from the top of the head 350 through the distal end 320 of the shaft 310. There may be a tool engagement feature 306 at the top of the cannula 304 along the flat top surface 354 of the head 350. When the secondary fastener 300 is disposed in the head 250 of the primary fastener 300, the cannula 304 and the tool engagement feature 306 may be accessible through the first end 256 of the passageway 252. Thus, a guidewire may be inserted from the first end 256 of the passageway 252 through the cannula 306 of the secondary fastener 300. Moreover, a tool, like for example a screwdriver, may be able to engage the tool engagement feature 306 of the secondary fastener 300 through the first end 256 of the passageway 252 to implant, or screw, the secondary fastener 300 into the bone. In some embodiments, the guidewire may be inserted through the cannula 304 of the secondary fastener 300 and inserted into the desired location in the vertebra. Then, the shaft 310 of the secondary fastener 300 may be implanted around the guidewire using a tool that engages the tool engagement feature 306. In some embodiments, the tool engagement feature 306 may be hexagonally or hexalobe shaped. However, in other embodiments, the tool engagement feature 306 may be any other appropriate shape including, triangular, square, rectangular, ovular, or pentagonal.

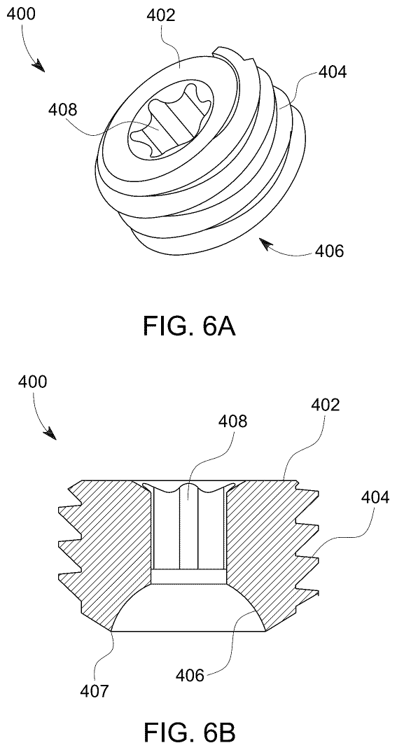

FIGS. 6A and 6B illustrate a perspective view and a cross-sectional view, respectively, of the set screw 400 of the bone fixation system 100 shown in FIG. 2. After secondary fastener 300 is positioned into bone in a selected direction and orientation relative to the primary fastener 200, set screw 400 may lock the orientation of secondary fastener 300 relative to the primary fastener 200. Set screw 400 may have any suitable size, configuration and means for securing the head 350 of the secondary fastener 300 to seat 260 in the passageway 252 of the primary fastener 200. The set screw 400 may include a top surface 402, side surface 404 and a bottom surface 406. Set screw 400 may also include a tool engagement feature 408 along the top surface 402. In some embodiments, the tool engagement feature 408 may be hexagonally or hexalobe shaped. However, in other embodiments, the tool engagement feature 408 may be any other appropriate shape including, triangular, square, rectangular, ovular, or pentagonal. In some embodiments, set screw 400 may be cannulated and in other embodiments, it may not be cannulated. The set screw 400 may include threads 410 on side surface 404 extending partially or entirely from the top surface 402 to the bottom surface 406.

The bottom surface 406 may be sized and shaped to retain the head 350 of the secondary fastener 300 against the seat 260 of the passageway 252. In some embodiments, the shape of the bottom surface 406 may be curved to match the shape of the head 350 of the secondary fastener 300. In some aspects, the shape of the bottom surface 406 may maximize surface contact and, therefore, maximize the rigidity of the system. In some other embodiments, the radius of curvature of the bottom surface 406 may be smaller than the spherical radius of curvature of the head 350 of the secondary fastener 300. In the scenario where the geometry of the bottom surface 406 of the set screw 400 is smaller than the surface geometry of the head 350 of the secondary fastener 300, such a difference may result a secure engagement between the head 350 and the bottom surface 406 by the engagement (e.g., cutting into) of the lower circumferential break edges 407 of the set screw 400 with the surface of the head 350 when the set screw 400 is threaded into the head 250 of the primary fastener 200. In another embodiment, the bottom surface 406 may include a surface treatment, such as, for example, surface etching, which engages and/or interacts with head 350 of second fastener 300 to provide, for example, enhanced rigidity through a friction fit that prevents shifting of second fastener 300 once secured. In one example, head 350 may also include a surface treatment or similar surface etching that engages and/or interacts with bottom surface 406 of the set screw 400.

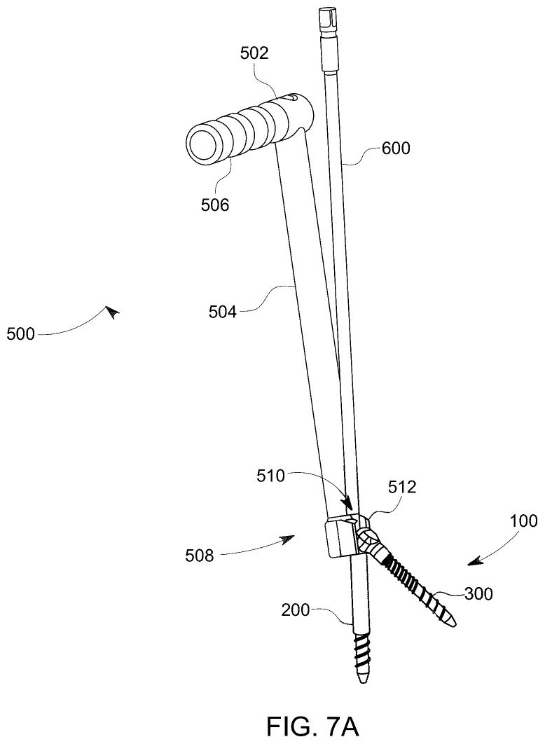

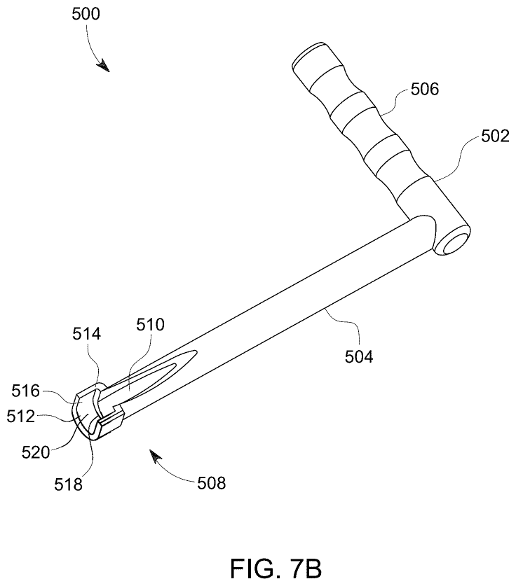

FIGS. 7A and 7B illustrate an inserter instrument 500 for implanting the bone fixation system 100 into bone. The inserter instrument 500 may have a handle 502 and a shaft 504 that extends from the handle 502. In some embodiments, the handle 502 may have grips 506 that allow a surgeon to hold and maneuver the inserter instrument 500 more easily. The bottom of the shaft 504 may have an attachment feature 508 for coupling to the bone fixation system 100. The attachment feature may have an opening or cutout 510 with a U-shaped or C-shaped interior surface 512. The interior surface 512 may have a ridge 514 spaced from the bottom end of the shaft. The interior surface 512 may have a first side surface 516 and a second side surface 518 connected by a curved surface 520. The side surfaces 516, 518 of the attachment feature 508 may contact and hold the side surfaces 278, 280 of the exterior surface 264 of the head 250 of the primary fastener 200. In some embodiments, the top surface 270 of the head 250 may contact the bottom of the ridge 514 of the attachment feature 508 of the inserter instrument 500. In this way, the surgeon may use the inserter instrument 500 to hold the primary fastener 200 steady as other instruments are used to implant the bone fixation system 100 into bone. For example, a screwdriver instrument 600 may be inserted along the opening 510 to contact the tool engagement feature 206 of the primary fastener 200 or the tool engagement feature 306 of the secondary fastener 300 to implant the respective fastener into bone.

In some embodiments, the bone fixation system 100 may be provided as a kit with one or more primary fastener 200 sizes and one or more secondary fastener sizes 300. In some embodiments, the primary fastener 200 and the secondary fastener 300 may be the same size. In other embodiments, the primary fastener 200 and the secondary fastener 300 may be different sizes. The surgeon may choose the primary fastener 200 and the secondary fastener 300 based on the patent's physiology or the surgeon's preference.

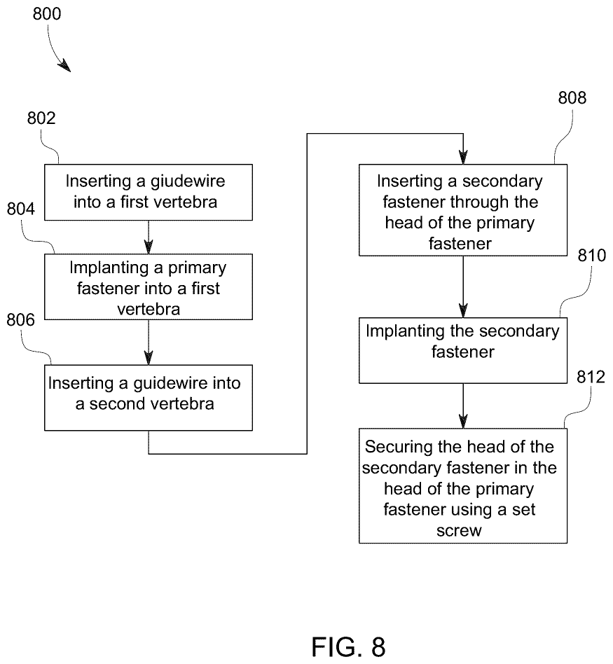

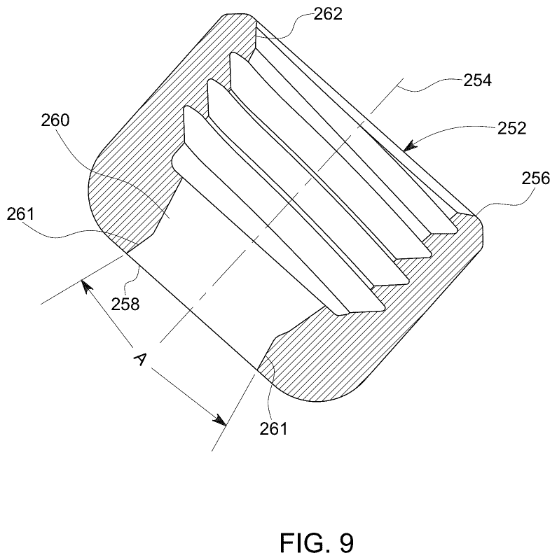

FIG. 8 illustrates a flow chart of a method 800 for implanting a bone fixation system 100 into a spine of a patient, according to some embodiments of the present disclosure. In some embodiments, the bone fixation system 100 may be implanted into the spine 50 of a patient to stabilize one or more levels of the spine 50. The method may include choosing a primary fastener 200 from a kit.

Step 802 of the method 800 may optionally include inserting a guidewire into a first vertebra 52 to a desired location. The guidewire may then be threaded through the cannula 204 of the primary fastener 200.

Step 804 of the method 800 may include implanting the primary fastener 200 into the first vertebra 52. If a guidewire is used, the primary fastener 200 may be implanted around the guidewire. A screwdriver may be used to screw the primary fastener 200 into the first vertebra 52. In some embodiments, the screwdriver may engage the tool engagement feature 206 through the first end 256 of the passageway 252 to implant the primary fastener 200 into bone. In some embodiments, the screwdriver is cannulated so that a guidewire may be inserted through the primary fastener 200 and through the screwdriver. Once the primary fastener 200 is implanted, the guidewire may be removed.

Step 806 of the method 800 may optionally include inserting a guidewire into a second vertebra 54 to a desired location. The guidewire may then be threaded through the cannula 304 of the secondary fastener 300.

Step 808 of the method 800 may include inserting the secondary fastener 300 through the passageway 252 of the head 250 of the primary fastener 200. The shaft 310 of the secondary fastener 300 may be inserted first through the passageway 252 until the head 350 is seated within the passageway 252. In some embodiments, the curved surface 352 of the secondary fastener 300 will contact the seat 260 in the passageway 252. The secondary fastener 300 may pivot about the seat 260 to the desired angle.

In some embodiments, an angle guide may optionally be used to determine the appropriate trajectory of the secondary fastener 300. The angle guide may have a curved end or a spherical ball shape that matches or is smaller than the curved bottom surface 352 of the head 350 of the secondary fastener 300. The angle guide may have an inner lumen that passes through the curved end of the angle guide. The angle guide may fit through the first end 256 of the passageway 252 of the head 250 of the primary fastener 200 and may contact the seat 260. The curved surface or ball end of the angle guide may pivot about the seat 260 similar to the way the secondary fastener 300 pivots about the seat 260. Thus, the surgeon may use the angle guide to determine the desired angle of the secondary fastener 300 using the angle guide. Once the desired angle is determined, the surgeon may insert a guidewire through the lumen of the angle guide and insert the guidewire into the second vertebra 54 at the desired angle. The angle guide may then be removed. In some embodiments, the secondary fastener 300 is implanted without the use of an angle guide.

Step 810 of the method 800 may include implanting the secondary fastener 300 into the second vertebra 54. A screwdriver may be used to implant the secondary fastener 300 into the bone. The screwdriver may engage the tool engagement feature 306 on the head 350 of the secondary fastener 300 through the first end 256 of the passageway 252 of the primary fastener 200 to screw the secondary fastener 300 into the bone around the guidewire. In some embodiments, the screwdriver is cannulated so that a guidewire may be inserted through the secondary fastener 300 and through the screwdriver. Once the secondary fastener 300 is implanted, the guidewire may be removed.

In some embodiments, an inserter instrument 500 may be used to aid in implanting the secondary fastener 300 into the bone. The head 250 of the primary fastener 200 may be inserted into the attachment feature 508 of the inserter instrument 500. The opening 510 of the attachment feature 508 of the inserter instrument 500 may be placed around the head 250 of the primary fastener 200 such that the ridge 514 rests on the top surface 270 of the head 250. A screwdriver instrument 600 may then be inserted into the first end 256 of the passageway 252 of the primary fastener 200 to contact the tool engagement feature 306 on the head 350 of the secondary fastener 300. The inserter instrument 500 may then be held steady as the screwdriver instrument 600 is used to implant or screw in the secondary fastener 300.

Step 812 of the method 800 may also include securing the head of the secondary fastener 300 in the head 250 of the primary fastener 200 using a set screw 400. In some embodiments, the set screw 400 may have threading along the side surface 404 that may engage a threaded portion 286 of the passageway 252. The set screw 400 may be inserted into the first end 256 of the passageway 252 of the primary fastener 200. In some embodiments, a screwdriver may be used to screw the set screw 400 into the passageway 252 by engaging the tool engagement feature 408 on the top of the set screw 400. The set screw 400 may contact the head 350 of the secondary fastener 300 and secure it against the seat 260 of the passageway 252. Thus, the set screw 400 may be tightened until it retains the secondary fastener 300 in the same angular position relative to the primary fastener 200 and prevents the secondary fastener 300 from moving.

In some embodiments, the inserter instrument 500 may be used to aid in inserting and tightening the set screw 400 into the passageway 252 of the primary fastener 200. The head 250 of the primary fastener 200 may be inserted into the attachment feature 508 of the inserter instrument 500 as described above. A screwdriver instrument 600 may then be inserted into the tool engagement feature 408 of the set screw 400. The inserter instrument 500 may then be held steady as the screwdriver instrument 600 is used to insert and tighten the set screw 400 in the passageway 252.

In some embodiments, a drill instrument may be used to drill a hole before the fasteners 200, 300 are implanted. This may make it easier for the surgeon to implant the fasteners 200, 300. In some embodiments, alternative methods may be employed to install the bone fixation system 100 described herein. For example, a surgical robot or an image guidance system may be used to perform or aid in performing all or portions of the various steps required to properly install a system constructed in accordance with one or more aspects within a patient.

Although the fasteners shown and described through the present description, such as fasteners 200 and 300 are shown as being straight, it is also contemplated that one or more of these fasteners may have curved shafts that may be driven into the patient's vertebrae rather than screwed, or the shafts of these fasteners may also be slightly curved.

As may be recognized by those of ordinary skill in the art based on the teachings herein, numerous changes and modifications may be made to the above-described and other embodiments of the present disclosure without departing from the scope of the disclosure. The fasteners, elongate members, and other components of the devices and/or systems as disclosed in the specification, including the accompanying abstract and drawings, may be replaced by alternative component(s) or feature(s), such as those disclosed in another embodiment, which serve the same, equivalent or similar purpose as known by those skilled in the art to achieve the same, equivalent or similar results by such alternative component(s) or feature(s) to provide a similar function for the intended purpose. In addition, the devices and systems may include more or fewer components or features than the embodiments as described and illustrated herein. For example, the components and features of FIGS. 1-7B and FIG. 9 may all be used interchangeably and in alternative combinations as would be modified or altered by one of skill in the art. Accordingly, this detailed description of the currently-preferred embodiments is to be taken as illustrative, as opposed to limiting the disclosure.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The disclosure has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the disclosure be construed as including all such modifications and alterations.

What is claimed is:

1. A bone fixation system, comprising:
a first fastener comprising:
a first shaft comprising a distal end, a proximal end, a first longitudinal axis, and a first neck disposed at the proximal end, wherein the first neck is unthreaded;
a first head disposed at the proximal end of the first shaft and defining a passageway extending from a first end to a second end and having a passageway axis, the first head comprising:
an interior surface along the passageway having a passageway axis and having a curved seat, the first longitudinal axis and the passageway axis being obliquely angled;
a front surface disposed between the first neck of the first shaft and the first end of the first head, wherein the front surface is generally parallel to the first longitudinal axis;
a U-shaped back portion intersecting a first edge and a second edge of the front surface, wherein the U-shaped back portion comprising a first side surface, a second side surface, and a curved surface connecting the first and second side surfaces, wherein the first and second side surfaces are generally planar and are generally aligned with the passageway axis,
a second fastener comprising:
a second shaft comprising a distal end, a proximal end, a second longitudinal axis, and a second neck disposed at the proximal end, wherein the second neck is unthreaded;
a second head disposed at the proximal end of the second shaft, the second head having a curved surface sized and shaped to contact the seat of the first head and pivot about the curved surface; and
a set screw introducible into the passageway along the passageway axis to secure the second head of the second fastener in the passageway between the seat and the set screw and to lock the second fastener in a fixed, non-pivotable position.

2. The bone fixation system of claim 1, wherein the first edge between the first side surface and the front surface is rounded and wherein the second edge between the second side surface and the front surface is rounded.

3. The bone fixation system of claim 1, wherein the first fastener comprises threads between the unthreaded first neck and the distal end.

4. The bone fixation system of claim 3, wherein the threads of the first fastener comprise a first portion and a second portion, wherein the first portion has a higher thread pitch than the second portion.

5. The bone fixation system of claim 3, wherein an intersection between the first neck and the threading on the first shaft is at least one of rounded or tapered.

6. The bone fixation system of claim 1, wherein the second fastener is threaded between the unthreaded second neck and the distal end.

7. The bone fixation system of claim 1, wherein the second longitudinal axis is parallel to the passageway axis.

8. The bone fixation system of claim 1, wherein the second longitudinal axis is obliquely angled relative to the passageway axis.

9. The bone fixation system of claim 1, wherein the second fastener is pivotable such that the second longitudinal axis is angled in a range of 0 degrees to 15 degrees relative to the passageway axis.

10. A bone fixation system, comprising:
a first fastener comprising:
a first shaft comprising:
a threaded distal end;
a proximal end;
a first longitudinal axis; and,
a first neck disposed at the proximal end, wherein the first neck is unthreaded;
a first head disposed proximal the neck and defining a passageway extending between a first passage end and a second passage end and having a passageway axis obliquely angled relative to the first longitudinal axis, the first head comprising a seat disposed along the passageway,
wherein an outer profile of the first head does not match an inner profile of the first head, and
wherein the inner profile is cylindrical and the outer profile comprises at least one flat surface;
a second fastener comprising:
a second shaft comprising:
a distal end;
a proximal end;
a second longitudinal axis; and,
a second neck disposed at the proximal end, wherein the second neck is unthreaded;
a second head disposed proximal the second neck, the second head being bulbous and having a curved surface, wherein the second head is shaped for disposal within the passageway such that the curved surface of the second head contacts the seat of the first head and is pivotable about the curved surface; and
a set screw introducible into the passageway along the passageway axis to secure the second head of the second fastener in the passageway between the seat and the set screw and to lock the second fastener in a fixed, non-pivotable position.

11. The bone fixation system of claim 10, wherein the outer profile comprises a a front surface disposed between the first neck of the first shaft and a top surface of the first head; and,
a U-shaped surface connecting each side of the front surface, wherein the U-shaped surface comprises a first side surface, a second side surface, and a curved surface connecting the first and second side surfaces,
wherein at least one of the front surface, the first side surface, and the second side surface is the at least one flat surface.

12. The bone fixation system of claim 11, wherein a first edge between the first side surface and the front surface is rounded and wherein a second edge between the second side surface and the front surface is rounded.

13. The bone fixation system of claim 10, wherein the first fastener is threaded between the unthreaded first neck and the distal end.

14. The bone fixation system of claim 13, wherein an intersection between the first neck and the threading on the first shaft is at least one of rounded or tapered.

15. The bone fixation system of claim 10, wherein the second longitudinal axis is parallel to the passageway axis.

16. The bone fixation system of claim 10, wherein the second longitudinal axis is obliquely angled relative to the passageway axis.

17. The bone fixation system of claim 10, wherein the second fastener is pivotable such that the second longitudinal axis is angled in a range of 0 degrees to 15 degrees relative to the passageway axis.

18. A method of implanting a bone fixation system, comprising the steps of:
introducing a first fastener into a first bone, the first fastener comprising:
a first shaft comprising:
a distal end;
a proximal end;
a first longitudinal axis; and
a first neck disposed at the proximal end, wherein the first neck is unthreaded;
a first head disposed at the proximal end of the first shaft and defining a passageway extending from a first end to a second end and having a passageway axis, the first head comprising:
an interior surface along the passageway, wherein the interior surface defines a curved seat;
a front surface disposed between the first neck of the first shaft and the first end of the first head, wherein the front surface is generally aligned with the first longitudinal axis;
a U-shaped surface connecting each side of the front surface, wherein the U-shaped surface comprises a first side surface, a second side surface, and a curved surface connecting the first and second side surfaces, wherein the first and second side surfaces are generally planar and are generally aligned with the passageway axis,
wherein the first longitudinal axis and the passageway axis are obliquely angled;
introducing a second fastener into a second bone, second fastener comprising:
a second shaft comprising:
a distal end;
a proximal end;
a second longitudinal axis; and
a second neck disposed at the proximal end, wherein the second neck is unthreaded;
a second head disposed at the proximal end of the second shaft, the second head being a bulbous head and having a curved surface, wherein the second head is shaped for disposal within the passageway such that the curved surface of the second head contacts the seat of the first head and is pivotable about the curved surface;
implanting the first fastener;
implanting the second fastener through the passageway at a first angle, wherein the first angle is one of: parallel to the passageway axis or obliquely angled relative to the passageway axis; and,
securing the second head of the second fastener in the passageway using a set screw to lock the second fastener in a fixed, non-pivotable position.

19. The method of claim 18, wherein a first edge between the first side surface and the front surface is rounded and wherein a second edge between the second side surface and the front surface is rounded.

20. The bone fixation system of claim 1, wherein the first fastener further comprises:
a threaded portion between the unthreaded first neck and the distal end; and,
an intersection between the first neck and the threaded portion, wherein the intersection is at least one of rounded or tapered.

21. A bone fixation system, comprising:
a first fastener having a proximal end and a distal end, the first fastener comprising:
a first shaft defining a first longitudinal axis; and
a first head disposed at the proximal end of the first shaft, the first head comprising:
a passageway sized to receive a second fastener, the passageway having a passageway axis obliquely angled relative to the first longitudinal axis, the passageway having an interior surface defining a curved seat shaped to interface with the second fastener;
a front surface forming a plane generally parallel to the first longitudinal axis;
a U-shaped back portion extending from opposing sides of the front surface, wherein the U-shaped back portion comprises a first planar side surface, a second planar side surface, and a curved surface connecting the first and the second side surfaces, the first side surface and the second side surface being generally planar and generally parallel to the passageway axis, the curved surface defining a reference line angled relative to and intersecting a reference line along the front surface.

22. The bone fixation system of claim 1, wherein a first edge between the first side surface and the front surface is rounded and wherein a second edge between the second side surface and the front surface is rounded.
